(19) United States
(12) Patent Application Publication
(10) Pub. No.: US 2004/0031070 A1
(43) Pub. Date: Feb. 12, 2004

(54) RNA transfection of sperm cells

(76) Inventors: Corrado Spadafora, Rome (IT); Clague Pitman Hodgson, Omaha, NE (US); Rodolfo N. Lorenzini, Rome (IT)

Correspondence Address: Clague P. Hodgson, Nature Technology Corp., 4701 Innovation Drive, Lincoln, NE, 68521, US

(21) Appl. No.: 09/977,179

(22) Filed: Oct. 15, 2001

(51) Int. Cl. A01K 67/027; C12N 15/86

(52) U.S. Cl. 800/21; 435/456

(57) ABSTRACT

A method is described for transferring expressible foreign genes into multi-cellular organisms by means of RNA uptake by sperm cells, followed by endogenous reverse transcription of RNA into cDNA. cDNA molecules are transferred to embryos through fertilization, and are transmitted to born animals and their progeny. The cDNA sequences are mosaically distributed in the cells and tissue of the organisms, and are preferentially expressed in target tissues.

### CROSS-REFERENCE TO RELATED APPLICATIONS

Provisional patent application by the same title and inventors, filed in The US Patent and Trademark Office, Oct. 13, 2000.

Literature References:

US Patent Documents

Freeman S, Abraham G, McCune C S, Moolten F L, Koeplin D. Cancer therapy utilizing malignant cells expressing HSV-TK (1997), U.S. Pat. No. 5,601,818.

Scientific Literature

Hogan B, Beddington R, Costantini F, Lacy E. Manipulating the mouse embryos: a laboratory manual. (1994) Cold Spring Harbor Laboratory Press, Cold Spring Harbor, N.Y.

Jaenisch R. Germ line integration and mendelian transmission of the exogenous Moloney leukemia virus. (1976) Proc. Natl. Acad. Sci. (USA) 73: 1260-1264

Lavitrano M, Cameron A, Fazio V M, Dolci S, Farace M G, Spadafora C. Sperm cells as vectors for introducing foreign DNA into eggs: genetic transformation of mice. (1989) Cell. 57: 717-23.

Palmiter R D, Brinster R L. Germ-line transformation of mice. (1986) Annu. Rev. Genet. 20: 465-99.

Salter D and Crittenden L B Artificial insertion of a dominant gene for resistance to avian leukosis virus into the germ line of the chicken. (1989) Theor. Appl. Genet. 77: 457-461

Hodgson C P and Solaiman F. Virosomes: Cationic liposomes enhance retroviral transduction. (1996) Nature Biotechnology 14:339-42.

Whittingham, D G, Culture of mouse ova. (1971) J. Reprod. Fertil. Suppl. Jun;14:7-21.

International Patent Documents

Hodgson C P, Zink M A, Xu G-P. Chimeric Viral Packaging Signal without Gag Gene Sequences. (1998) PCT/US98/24667.

### BACKGROUND

1. Field of Invention

This invention relates to methods and materials for the production and use of transgenic multi-cellular organisms in agriculture and biotechnology, as well as for gene therapy in man.

2. Description of the Prior Art

Transgenic plants and animals result from the introduction of foreign nucleic acids into the germ line. The nucleic acids (RNA or DNA) can be introduced in the form of either a virus (such as a retrovirus), so-called transduction, or through direct introduction by physical or chemical means (transfection).

One of the first examples of germ line transduction was Jaenisch, 1976, who incubated murine retroviruses with mouse blastocysts, creating mice derived from both transgenic and non-transgenic cells (chimeric mice). The chimeric mice were then bred to produce lines of mice with unique insertions of proviruses in the germ line. Later, genetically engineered retroviral vectors were used to make transgenic mammals and avians (Salter and Crittenden, 1989).

Early examples of germ line transfection used micro-injection to introduce DNA into the male pronucleus of fertilized zygotes (Palmiter and Brinster). Other methods of transfection include: electroporation, gene gun, and chemical introduction (by lipofection, calcium phosphate, DEAE dextran, etc.)

An unusual method for introducing genes into animals by sperm transfection was first reported in 1989 by Lavitrano and Spadafora (Lavitrano et al., 1989). Briefly, mouse epididymal spermatozoa were pre-incubated with pSV2CAT plasmid DNA and used in in vitro fertilization assays. Plasmid sequences were found in about 30% of the F₀, animals generated with these fertilizations and some of them expressed the CAT gene; foreign DNA sequences were further transmitted to the F₁ progeny when founders were bred with wild type mice.

Although a variety of methods are available for introducing genes into the germ lines of multi-cellular organisms, all share the common feature that they are somewhat technically difficult. Ideally, it would be desirable to have a method for introducing foreign genes directly into the animal in a manner that was both heritable and straight-forward. It would be especially desirable if the method provided episomal transmission (as opposed to integration into the germ line), in order to preserve germ line integrity. This would be particularly useful in human gene therapy procedures, where germ line integration should be avoided.

In the instant invention, RNA is taken up by the sperm, where it is reverse-transcribed into cDNA and is faithfully expressed and transmitted to the organs and tissues of founder animals and their progeny.

### SUMMARY

In accordance with the present invention, transgenic animals may be obtained by fertilizing eggs with sperm that has been incubated with RNA containing one or more transgenes.

### Objects and Advantages

Accordingly, besides the ease and directness by which one may obtain transgenic animals in our above patent, several objects and advantages of the present invention are:

(a) To provide a means to treat hereditary diseases and cancer;

(b) to provide episomal transmission of the transgene(s), without disrupting the chromosomal DNA;

(c) to provide active expression of transgenes in the animal(s);

(d) to provide replication of the transgenes;

(e) to provide a means of removing the transgenes from the animals at a later time; and

(f) to provide a means of marking particular strains of animals to prevent theft of proprietary strains.

Further objects and advantages will become apparent from a consideration of the ensuing description and drawings.

### DRAWING FIGURES

FIG. 1. Schematic outline of the procedure for generating animals expressing new genes by means of RNA transfer.

FIG. 2. Schematic drawing of the vector VLMB.

FIG. 3. Table summarizing the results of polymerase chain reaction (PCR) analysis of cDNA in various tissues of F₀ offspring.

FIG. 4. Detailed example of an analysis of PCR testing (two animals).

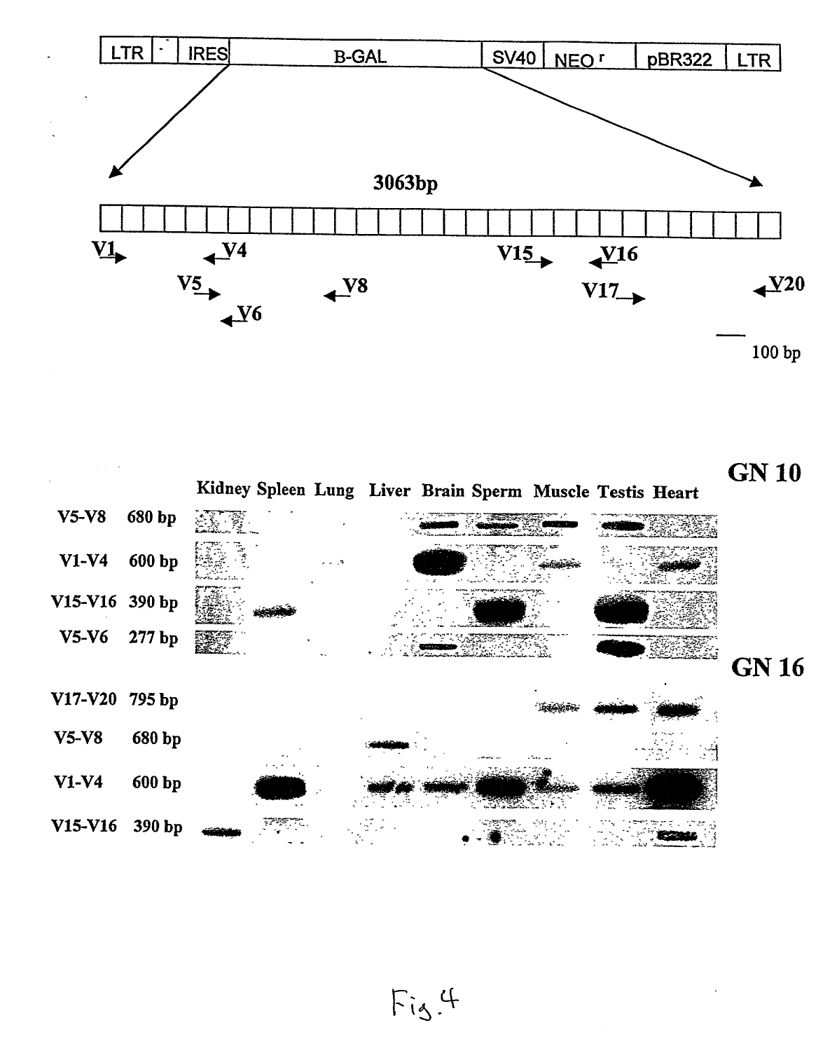

FIG. 5. Table summarizing the results of polymerase chain reaction (PCR) analysis of cDNA in various tissues of F₁ offspring.

### DESCRIPTION—DEFINITIONS

Definitions

Reverse transcription: The process by which ribonucleic acid (RNA) molecules are converted into deoxyribonucleic acid molecules (DNA).

Retrovirus: Any of the family of viruses known as retroviridae. A characteristic of these viruses is their ability to convert RNA molecules to DNA molecules by means of reverse transcriptase.

Retrotransposon(s): Cellular mobile genetic element(s) that replicate by means of the enzyme reverse transcriptase, either by means of a retrotransposon-encoded reverse tranxcriptase enzyme, or by means of a viral or cellular reverse transcriptase enzyme.

Retro-vector: A vector for gene transfer that is derived from a retrovirus or retrotransposon.

Transfection: Transfer of RNA or DNA into cells, tissues, or embryos by chemical or physical methods.

Transduction: Transfer of RNA or DNA into cells, tissues, or embryos by means of virus particles.

Founder animal: The first generation of animal that contains a foreign gene that is transmissible to its progeny.

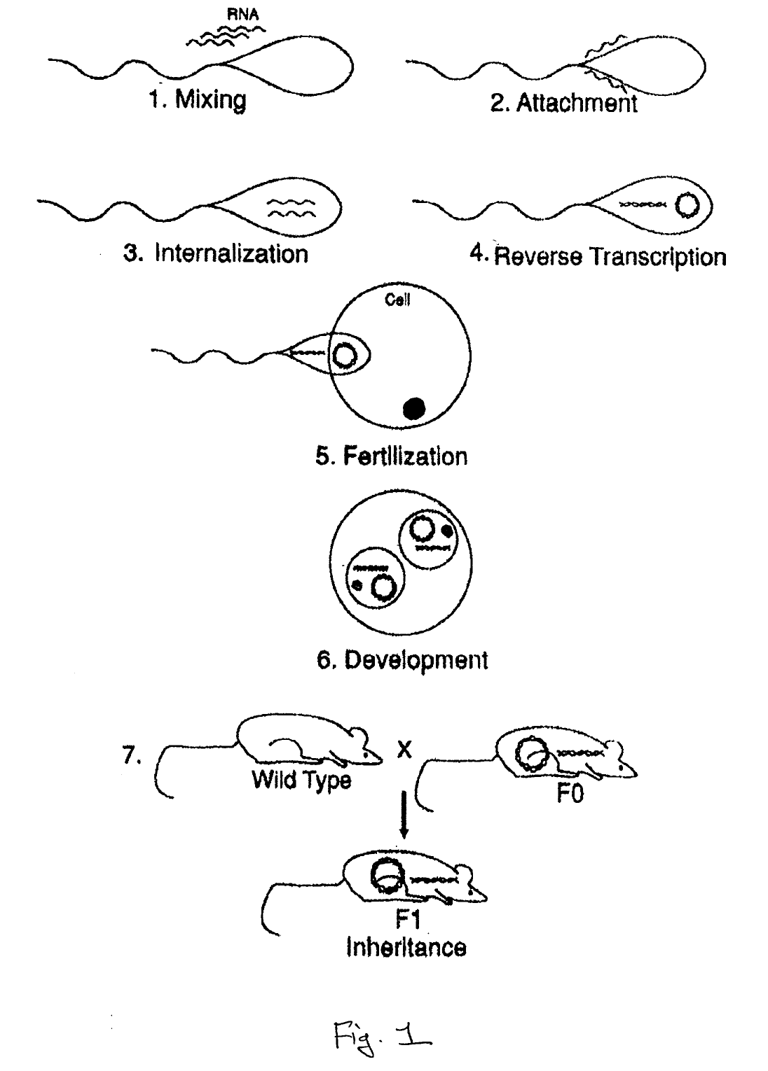

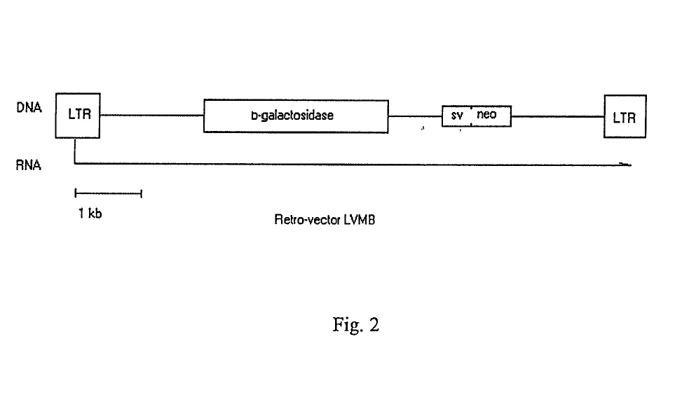

### DESCRIPTION—FIGS. 1 AND 2—PREFERRED EMBODIMENT

In the invention (FIG. 1), the RNA molecules are incubated with and are taken up by the sperm cells, where they are reverse-transcribed into complementary DNA (cDNA) molecules. The sperm cells are used to fertilize oocytes in vitro, transferring to the embryos the newly synthesized cDNA. The two-cell embryos are transferred to foster mothers, where they give rise to founder (F₀)) animals. Analysis of the F₀ animals indicates distribution and expression in different tissues. Through breeding, the cDNA sequences can be transmitted and expressed in subsequent generations of animals.

In a preferred embodiment of the invention, the RNA molecules are derived from a retrovirus, a retrotransposon, or a retro-vector derived from sequences related to retroelements, such as the exemplary vector VLMB (FIG. 2). Such RNA molecules favor the uptake and conversion of the RNA to linear and circular cDNA molecules that are substrates for potential integration (into genomic DNA) as well as for autonomous replication as episomal DNA molecules.

Preferred Embodiments of the Invention

Preferred Embodiment 1: In vitro fertilization (IVF) generation of F₀ founder mice expressing new genetic information using spermatozoa pre-incubated with RNA.

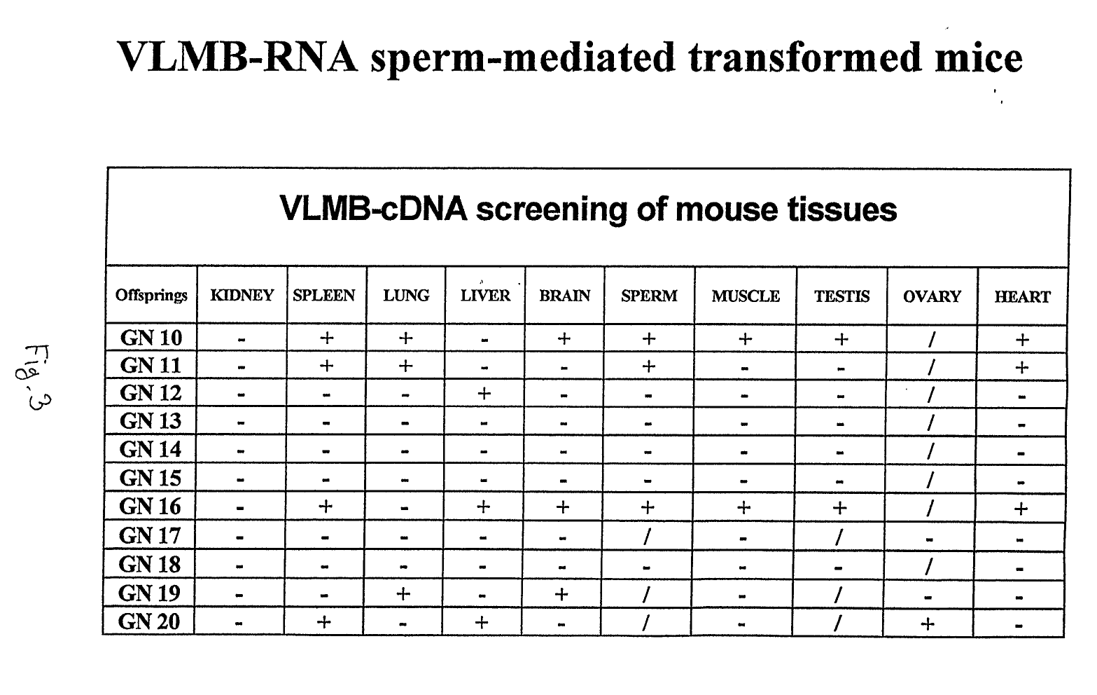

Mouse epididymal sperm cells were surgically obtained from male donor(s) (Hogan, 1994) and incubated with RNA transcribed from retroviral vector pVLMB DNA (Hodgson et al., PCT/US98/24667) bearing the Lac-z (beta-galactosidase) reporter gene. Routinely, 50 ng of RNA per 10⁶ spermatozoa were mixed in fertilization medium (FM, Whittingham, 1971) for 30 min in 5% carbon dioxide in air. 2×10⁶ sperm cells were then withdrawn from the incubation mixture and used to fertilize oocytes according to published procedure (Hogan, 1994); embryos were grown in vitro to the two-cell stage and then implanted into foster mothers. Approximately 30 days after birth, randomly selected offspring were sacrificed, DNA samples were extracted from different organs, and were analyzed by direct PCR using appropriate oligonucleotide primer combinations designed to amplify portions of the Lac-z gene (FIGS. 3 & 4). The results demonstrated that: a) cDNA molecules complementary to the sperm-challanging RNA were detected in most of the screened animals; b) the cDNA was distributed non-homogeniously within the organs of the same animal, including gonads and gametes; and c) Lac-z gene expression was detected by appropriate staining in some of the PCR-positive organs. Preferred Embodiment 2: Foreign genetic information is transferred from F₀ founders to F₁ progeny.

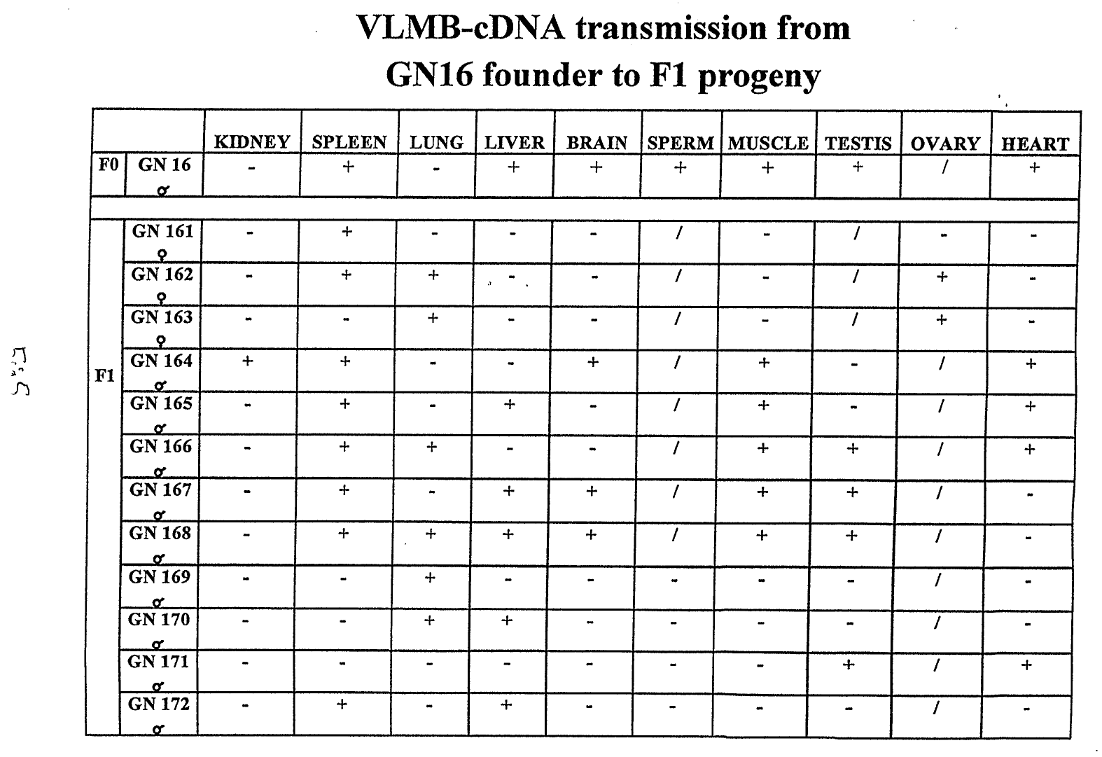

Founders were bred with wild type mice generating F₁ progeny. Offspring were preferentially selected from those generated by founders that had PCR-positive gonads and gametes. F₁ animals were sacrificed and the DNA was extracted from various organs and was analyzed by direct PCR amplification as described above (FIG. 5). The results demonstrate that: a) cDNA molecules are transferred from F₀ founders to F₁ progeny via gametes; b) cDNA was detected with variable frequencies in all F₁ animals (at least in the DNA of one organ of each animal); c) the cDNA shows a mosaic distribution within the organs of the same animal; d) some organs, more than others, appear to be preferential targets for cDNA molecules; and e) the Lac-z retrogene shows a sharp preferential expression in specific tissues (spleen and lungs).

Preferred Embodiment 3: Foreign genetic Information is Transferred From Targeted tissues of positive mice to 3T3 cell cultures.

F₀ and F₁ mice were sacrificed, organs were collected, and DNA samples were extracted from a small fragment of each organ and used for PCR screening. PCR positive tissues were homogenized in a Potter homogenizer, spun down to sediment cellular debris and the supernatants were collected and filtered through a 0.22 μm filter. Aliquots of the supernatants were then incubated with Lipofectamine™ reagent (Invitrogen Corporation, Bethesda, Md.) as described by Hodgson and Solaiman, 1996, Supra, and were then added to mouse NIH 3T3 cell cultures and incubated overnight. The following day the supernatant-containing medium was removed and substituted with fresh medium and cells were allowed to grow for one more day. 0.8 mg/ml of G418 were added to plates and cells were incubated for further 5 to 7 days. Control plates, containing cells that had not been incubated with supernatants, as well as those incubated with equivalent supernatants extracted from organs of wild type mice, were also incubated in parallel with the same amounts of G 418. Cells resistant to G418 were selected among the plates treated with supernatants from PCR positive tissues but not from untreated cells or those incubated with extracts from tissues of wild type mice. This result suggested that NIH 3T3 cells acquired G418 resistance through a transduction-like process, and that the foreign genes were transmitted by a retrovirus or a retrovirus-like particle.

Preferred Embodiment 4: Use of RNA to Prevent Manifestations of Hereditary Disease

A retro-vector, such as the exemplary VLMB, above, is adapted by the addition or substitution of a therapeutic gene, for example the cystic fibrosis membrane conductance regulator (CFTR) gene that plays a key role in cystic fibrosis disease (when the gene is defective). The compensatory gene may alternatively be: used alone; substituted for the reporter gene; or fused to the reporter gene. In a preferred embodiment of CFTR gene therapy, the CFTR gene promoter may be included in the construct, or another promoter, such as a retroviral long terminal repeat promoter, or retrotransposon long terminal repeat promoter (such as a retrotransposon VL30 long terminal repeat promoter) may be used to express the gene. The retro-vector DNA is then introduced into a retrovirus vector producer cell line, and vector RNA is produced and packaged into virions that are released into the cell culture medium. The vector RNA is then harvested from the virions by first concentrating the virus by centrifugation of the cell culture media from vector producer cells, then extracting the RNA from the virus by means of organic solvents. Briefly, for example, the cell culture media is removed from vector producer cells and is filtered through 0.45 micron cellulose acetate filters. The filtered media is then centrifuged, for example at 18,000 RCF for 30 min. to pellet the virus. The virus pellet is resuspended in 1 ml of phosphate buffered saline (PBS, Invitrogen Corp., Bethesda, Md.), and is extracted with an equal volume of phenol/chloroform/isoamyl alcohol (25:24:1). The aqueous phase is re-extracted twice with chloroform/isoamyl alcohol (24:1), and is precipitated with 1/9^{th} volume of sodium acetate, pH 5.6, and three volumes of ethanol.

50ng of the RNA/10⁶ sperm cells is then incubated with human sperm cells that have been washed as described in Preferred Embodiment 1, supra. The RNA is used to fertilize human eggs, using IVF or artificial insemination, resulting in offspring bearing, transmitting and expressing the transgene (refer to Preferred Embodiment 1 for details, as well as to Preferred embodiments 2 and 3 for additional information about detection and transmission). Expression of the transgene(s) in humans is detected by a method such as sweat chloride analysis, patch clamp studies, antibody detection, etc. Alternatively, the fusion reporter-therapeutic gene expression is observed by means of detection of the reporter gene.

Because the therapeutic gene is inserted episomally, it does not permanently alter the germ line. However, three caveats of human embryonic gene therapy are: 1) that the transgene might be regulated abnormally, requiring its removal; or 2) it might become integrated, requiring its removal at a later time; or 3) a better therapy (such as direct gene repair) may become available in the, requiring removal of the transgene. In any event, these exigencies may be addressed by including within the vector a so-called suicide gene, such as the herpes simplex virus thymidine kinase (TK) gene. The method of S. Freeman et al., (Cancer therapy utilizing malignant cells expressing HSV-TK, 1997, U.S. Pat. No. 5,601,818) can be used, and is incorporated herein as if fully set forth. Treatment of the human with a non-toxic pro-drug, such as gancyclovir, is used to eliminate cells expressing the transgene.

Although the description above contains many specificities, these should not be construed as limiting the scope of the invention but as merely providing illustrations of some of the presently preferred embodiments of this invention. Thus, the scope of the invention should be determined by the appended claims and their legal equivalents, rather than by the examples given.

We claim:

1) A method for introducing nucleic acid molecules into human beings and into non-human organisms comprising:
c) Isolating and purifying RNA molecules;
d) Incubating the RNA molecules with sperm cells;
e) Fertilizing eggs with the sperm cells;
f) Implanting the eggs or embryos derived from eggs into the appropriate host species and generating offspring; and
g) Identifying offspring expressing the desired traits of the introduced genes.

2) The method of claim 1, in which the RNA is derived from a retrovirus, a retrotransposon, or a retro-vector.

3) The method of claim 1, in which the RNA is derived from a cell or cells expressing the RNA as cellular, viral, or vector RNA.

4) The method of claim 1, in which a cell extract containing RNA or intracisternal virus-like particles is used in place of purified RNA molecules.

5) The method of claim 1, in which a cellular RNA is used.

6) The method of claim 1, in which the RNA contains a viral or retro-viral packaging signal.

7) The method of claim 1, in which the RNA contains viral integration sequences.

8) The method of claim 1, in which the RNA contains retroviral or retrotransposon long terminal repeats (LTRs).

9) The method of claim 1, in which the RNA molecule(s) are derived from in vitro-transcribed RNA, such as T7 or SP6 polymerase-derived RNA.

10) The method of claim 1, in which the resulting transgenic organisms are used for agricultural or biotechnological purposes.

11) The method of claim 1, in which the resulting organisms are used to produce proteins, polypeptides, antibodies, cytokines, hormones or antigens.

12) The method of claim 1, in which the resulting transgenic organisms are used to make nucleic acid molecules, RNA, DNA, anti-sense nucleic acid molecules, vectors (as RNA, DNA or virus particles) or triple helix nucleic acid molecules.

13) The method of claim 1, in which the resulting transgenic organisms are used to make proteins secreted as milk proteins or as egg proteins.

14) The method of claim 1, wherein artificial insemination is used in place of in vitro fertilization.

15) The method of claim 1, used to treat or to prevent a disease or to impart a different genotype or phenotype in a human being.

16) The method of claim 1, used to treat a disease or to impart a different phenotype in a human being, wherein the genes are transmitted episomally so as not to alter the germ line.

17) The method of claim 1, wherein the transmitted foreign genes further comprise a suicide gene, so as to permit removal of the transgene from the affected cells at a later time.

18) The composition of matter comprising at least one retrovirus-derived or retrovirus transposon-derived ribonucleic acid preparation, comprising at least: a) retroviral or retrovirus transposon long terminal repeats; b) reverse transcriptase primer binding sites; c) a retrovirus packaging signal; and d) at least one foreign gene, whereby the ribonucleic acid preparation enables reverse transcription and perpetuation of the RNA as DNA in a transgenic organism.

19) A method of gene transfer comprising:
a) generating an organism bearing foreign genes by the method of claims 1;
b) preparing a cell extract, virus preparation or RNA preparation from cells, organs, embryos or tissues of the organism;
c) incubating the extract with cells, tissues, or organs of another origin;
d) selecting for the expression of the transferred foreign gene(s) in the recipient cells.
